(19) United States
(12) Patent Application Publication
(10) Pub. No.: US 2017/0274036 A1
(43) Pub. Date: Sep. 28, 2017

(54) SYSTEMS, DEVICES, AND METHODS FOR SUBCUTANEOUS THERAPEUTIC TREATMENT

(71) Applicant: Boston Scientific SciMed Inc., Maple Grove, MN (US)

(72) Inventors: Javier Palomar-Moreno, Galway (IE); Michael Walsh, Galway (IE); Emma Jane Mooney, Co. Galway (IE); Damien Vincent Nolan, Galway (IE); Salman Musani, Galway (IE); Marie Turkington, Co. Mayo (IE)

(21) Appl. No.: 15/453,440

(22) Filed: Mar. 8, 2017

Related U.S. Application Data
(60) Provisional application No. 62/312,236, filed on Mar. 23, 2016.

(51) Int. Cl. A61K 38/00; A61K 35/28; B29C 67/24; A61K 9/08; A61K 9/00; A61K 9/50; A61K 31/7088

(52) U.S. Cl. CPC A61K 38/00; A61K 9/5031; A61K 35/28; A61K 31/7088; A61K 9/08; A61K 9/5089; A61K 9/0019; B29C 67/24; B29K 2995/006; B29L 2031/753

(57) ABSTRACT

An implantable medical device includes a body that has a flexible outer layer encapsulating a composition. The composition includes one or more first microspheres, one or more second microspheres, and a carrier. Each first microsphere includes a first therapeutic agent and a wall containing a first biodegradable polymer that encapsulates the first therapeutic agent. Each second microsphere includes a second therapeutic agent and a wall containing a biodegradable polymer that encapsulates the second therapeutic agent.

This application claims the benefit of U.S. Provisional Application No. 62/312,236, filed Mar. 23, 2016, the contents of which are herein incorporated by reference.

### TECHNICAL FIELD

This documents relates to systems, devices, and methods relating to controlled subcutaneous delivery and release of a therapeutic agent.

### BACKGROUND

Critical limb ischemia (CLI) is associated with severe obstruction of blood flow to a person's extremities (e.g., arms, legs, or feet) that has potential to eventually lead to limb loss. The symptoms associated with CLI can include pain in the foot at rest, non-healing ulcers, limb/digital gangrene, and delayed wound healing. An estimated 160,000 to 180,000 amputations are performed annually in the United States due to CLI. The rate of lower limb amputation in the United States has doubled since 1985 with a 4- to 5-fold increase in those over the age of 80. Fewer than half of all CLI patients may achieve full mobility after an amputation, and only one in four above-the-knee amputees will ever wear a prosthesis. The estimated cost of treating CLI is currently about 10 to 20 billion dollars per year in the US alone.

The quality of life for those with CLI can be extremely poor and reported to be similar to that of patients with end stage malignancy. Most patients with CLI may undergo repeat hospitalizations and surgical/endovascular procedures in an effort to preserve the affected limb(s). In certain circumstances, limb salvage efforts are not effective enough to reverse ischemia, and despite multiple attempts at revascularization, one or more wounds may fail to heal properly. In addition, many patients may not be eligible candidates for traditional forms of revascularization due to occluded or diffusely diseased distal vessels. Accordingly, there is a need in the art for therapies and devices that can treat critically ischemic limbs.

### SUMMARY

Disclosed herein are various embodiments of systems, devices, and methods relating to controlled subcutaneous delivery and release of a therapeutic agent. Advantages of the embodiments discloses herein include allowing the delivery an implantable device under the skin to release a therapeutic agent at a desired rate within the body. In some cases, certain embodiments provided herein can provide multiple release rates of one or more therapeutic agents from a single implantable device.

In Example 1, an implantable medical device includes a body that has a flexible outer layer encapsulating a composition. The composition includes one or more first microspheres, one or more second microspheres, and a carrier. Each first microsphere includes a first therapeutic agent and a wall containing a biodegradable polymer that encapsulates the first therapeutic agent. Each second microsphere includes a second therapeutic agent and a wall containing a biodegradable polymer that encapsulates the second therapeutic agent.

Example 2 includes implantable medical device of Example 1, wherein the body includes an oval-shaped or disc-shaped body.

Example 3 includes the implantable medical device of Example 1 or Example 2, wherein the outer layer of the body comprises a biodegradable polymer including polylactic acid (PLA), polyglycolic acid (PGA), a copolymer of PLA and PGA, poly-L-lactide (PLLA), poly-D,L-lactide (PDLA), poly-capralactone (PCL), or a combination thereof.

Example 4 includes the implantable medical device of any of Examples 1-3, wherein the carrier includes a liquid carrier.

Example 5 includes the implantable medical device of Example 4, wherein the liquid carrier includes water, saline solution, a serum, or a combination thereof.

Example 6 includes the implantable medical device of any of Examples 1-5, wherein the composition includes the first and second microspheres in an amount ranging from about 10% to about 90% by weight of the composition and the carrier in an amount ranging from about 90% to about 10% by weight of the composition.

Example 7 includes the implantable medical device of any of Examples 1-6, wherein the composition includes the first microspheres, the second microspheres, or a combination of both, in an amount ranging from about 10% to about 50% by weight of the composition, and the carrier in an amount ranging from about 50% to about 90% by weight of the composition.

Example 8 includes the implantable medical device of any of Examples 1-7, wherein the biodegradable polymer of the first microsphere generally degrades faster or slower than the biodegradable polymer of the second microsphere.

Example 9 includes the implantable medical device of any of Examples 1-8, wherein the biodegradable polymer of the first microsphere, the second microsphere, or both, includes polylactic acid (PLA), polyglycolic acid (PGA), a copolymer of PLA and PGA, poly-L-lactide (PLLA), poly-D,L-lactide (PDLA), poly-capralactone (PCL), or a combination thereof.

Example 10 includes the implantable medical device of any of Examples 1-9, wherein each of the first and second microspheres have a diameter ranging from about 0.2 millimeters to about 5 millimeters.

Example 11 includes the implantable medical device of any of Examples 1-10, wherein the walls of the first and second microspheres comprise a nonporous polymer layer.

In Example 12, an implantable medical device has a body that includes a first region and a second region. The first region contains a first carrier encapsulating one or more discrete locations containing a therapeutic agent within the first region. The second region contains a second carrier encapsulating one or more discrete locations containing a therapeutic agent within the second region, wherein the first carrier includes a first biodegradable material having a different degradation rate than the second carrier material, which contains a second biodegradable material.

Example 13 includes the implantable medical device of Example 12, wherein the first and second carriers include a gel, or a solid polymer matrix.

Example 14 includes the implantable medical device of Example 13, wherein the gel or the solid polymer matrix includes a biodegradable polymer including polylactic acid (PLA), polyglycolic acid (PGA), a copolymer of PLA and PGA, poly-L-lactide (PLLA), poly-D,L-lactide (PDLA), poly-capralactone (PCL), or a combination thereof.

Example 15 includes the implantable medical device of Example 12, wherein the first carrier has a degradation rate that is about 1.1, about 1.2, about 1.5, about 2, about 3, about 4, about 5, about 10, or about 20 times greater than a degradation rate of the second carrier.

Example 16 includes the implantable medical device of Example 12, wherein the body includes a total carrier composition that includes the first carrier in an amount ranging from about 40% to about 60% by weight of the total carrier composition and the second carrier in an amount ranging from about 40% to about 60% by weight of the total carrier composition.

Example 17 includes the implantable medical device of Example 12, wherein the composition includes the therapeutic agent in an amount ranging from about 10% to about 90% by weight of the composition and the first and second carriers in an amount ranging from about 90% to about 10% by weight of the composition.

Example 18 includes the implantable medical device of Example 12, wherein the therapeutic agent is selected from the group consisting of stem cells, adenoviruses, chemotherapeutic agents, immunosuppressants, proteins, nucleic acids, or a combination thereof.

In Example 19, a method of manufacturing an implantable device includes forming a first portion of a preformed body that includes a first biodegradable material and forming a second portion of the preformed body that includes a second biodegradable material, where the first portion degrades faster or slower than the second portion. The method further includes joining the first and second portions of the preformed body together to form the device and injecting a therapeutic agent into the first and second portions of the device. The method optionally includes encapsulating the therapeutic agent within the first and second portions of the device by sealing the injection sites created during the injecting step.

Example 20 includes the method of Example 19, wherein the forming of the first portion or the second portion comprises using one of injection molding, phase separation, emulsion or solvent evaporation, spraying, extrusion, sphere blowing, or 3D printing.

While multiple embodiments are disclosed, still other embodiments of the present disclosure will become apparent to those skilled in the art from the following detailed description, which shows and describes illustrative embodiments of the present disclosure. Accordingly, the drawings and detailed description are to be regarded as illustrative in nature and not restrictive.

### BRIEF DESCRIPTION OF THE DRAWINGS

FIG. 1 is a schematic view of an exemplary system provided herein that includes a subcutaneous implantable device for delivering a therapeutic agent into a patient's body.

FIG. 2 is a graph illustratively comparing two different exemplary microspheres (e.g., a fast-release microsphere and a slow-release microsphere) provided herein having different degradation times.

FIGS. 3A-3C are illustrations showing an exemplary method provided herein for subcutaneous implantation of the implantable device of FIG. 1

FIG. 4 is a schematic view of an alternative embodiment of an exemplary system provided herein that includes a subcutaneous implantable device for delivering a therapeutic agent into a patient's body.

FIGS. 5A-5H are schematic illustrations showing manufacturing steps provided herein for making the exemplary subcutaneous device of FIG. 4.

While the embodiments disclosed in this document are amenable to various modifications and alternative forms, specific embodiments have been shown by way of example in the drawings and are described in detail below. The intention, however, is not to limit this document to the particular embodiments described. On the contrary, this document is intended to cover all modifications, equivalents, and alternatives falling within the scope of the embodiments provided herein as defined by the appended claims.

### DETAILED DESCRIPTION

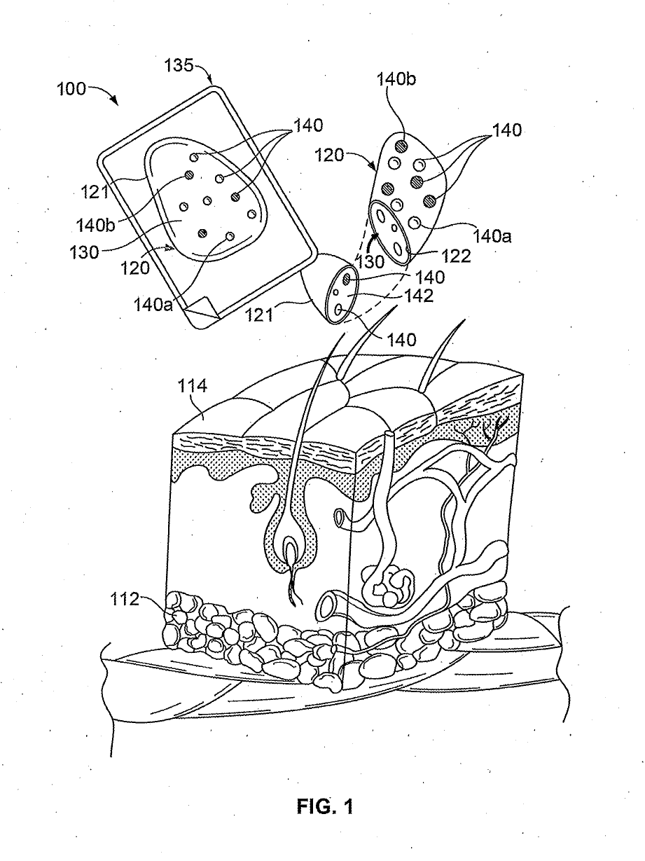

FIG. 1 is a schematic view of an exemplary system 100 for providing a sterile implantable device 120 containing a therapeutic composition 130 for implantation within a patient's subcutaneous region 112, according to various embodiments of the present disclosure. In some cases, the device 120 can deliver the therapeutic composition 130 within various locations of the patient's body, such as the legs or arms, the abdominal area, the thoracic area, or the cranial area of the patient's body. In some cases, the device 120 can be configured for delivering the composition 130 under the patient's skin 114 such that the device 120 resides within the epidermis or the dermis region. In some cases, the device 120 resides subcutaneously, that is, within or below the hypodermis (also referred to as the subcutis). The device 120 may be positioned, in some cases, between the fatty layer of the hypodermis and the muscular layer of the patient's tissue. The system 100 provided herein can be used for a wide range of medical applications for delivering the implantable device 120 and the composition 130 enclosed therein into the subcutaneous region 112 of the patient.

The system 100 of FIG. 1 includes the implantable device 120 enclosed within sterilizable packaging 135. The device 120 can include a body 121 that includes an oval-shaped or disc-shaped pouch that has a flexible outer layer 122 encapsulating the therapeutic composition 130. The device 120 can be inserted under the skin 114 (e.g., subcutaneously) through an opening created by an incision made at one or more desired locations along the patient's body, for example, the calf area of the patient's leg. The composition 130 provided herein may contain one or more therapeutic agents encapsulated within one or more microspheres 140 (which can also be referred to as microbeads). The therapeutic composition 130 may be encapsulated by the outer layer 122 of the body 121, which includes a biodegradable (or bioabsorbable) material adapted for degrading and thus releasing the microspheres 140 after a predetermined time (e.g., after one day of implantation, or after one week of implantation). Each microsphere 140 may contain a therapeutic agent and a wall comprising a biodegradable polymer that encapsulates the therapeutic agent. The biodegradable material of the wall of the microsphere 140 can be adapted to degrade and subsequent release the therapeutic agent contained therein into the patient's body after the microspheres have been released into the patient's body. Exposure to the enzymes within the patient's body can initiate the degradation of the body 121 and the microspheres 140 of the device 120. In some cases, the microspheres 140 can release the therapeutic agent (e.g., stem cells) at or near damaged tissue within the patient's anatomy to heal or treat the tissue.

As shown in FIG. 1, the sterilizable packaging 135 of the system 100 may include a sealable bag or envelope that is adapted to be peeled apart by a user. The packaging 135 may optionally include a tray (not shown) and/or a box (not shown). The packaging 135 can include various materials capable of withstanding a sterilization process, for example, a gamma radiation or ethylene oxide sterilization process. Suitable materials for a sterilized packaging can include, but are not limited to, high-density polyethylene and polypropylene.

The body 121 of the implantable device 120 can include the outer layer 122 containing the biodegradable material for temporally encapsulating the composition 130 containing microspheres 140. In some cases, the outer layer 122 of the device 120 can be made of various biodegradable materials, such as polylactic acid (PLA), polyglycolic acid (PGA), copolymers of PLA and PGA, poly-L-lactide (PLLA), poly-D,L-lactide (PDLA), poly-capralactone (PCL), and a combination thereof. In some cases, the outer layer 122 of the implantable device 120 can include a biodegradable polymer having a glass transition temperature that is greater than a patient's body temperature (e.g., a temperature at least equal to or greater than 45° C.).

The body 121 can be shaped in any desired form, for example, a polygonal shape such as a square, rectangular, triangular shape, or an irregular or asymmetrical shape. In some cases, the body 121 has any suitable outer dimensions, for example, a length and a width each ranging from about 5 mm to about 40 mm. The outer layer 122 of the body 121 can have a wall thickness configured for providing suitable biodegradable characteristics, for example, a degradation rate or an expected degradation (release) time. For example, in some cases, the outer layer 122 can have a wall thickness of about 0.5 mm to about 3 mm.

In some cases, the body 121 is configured to contain a liquid, gel, or solid medium, referred to as the “carrier,” 142 containing the therapeutic agent. Suitable liquid carriers include an aqueous-based solution or a non-aqueous (e.g., organic) solution. Exemplary liquid carriers include, but are not limited to, water (e.g., purified water, or distilled water), a saline solution, a serum (e.g., bovine serum or human serum albumin (HSA)), or a combination thereof.

In some cases, the carrier 142 can include a gel, such as a hydrogel. An exemplary carrier 142 can include a hyaluronic gel matrix, or the like. In some cases, the carrier 142 of the implantable device 120 can include a solid medium, such as a polymer matrix. In some cases, the carrier 142 can form the outer layer 122 of the body 121. The solid polymer matrix of the carrier 142 may, in some cases, include a biodegradable material, for example, the same or a similar polymer material as the outer layer 122 of the body 121. The implantable device 120 can include the gel or polymer matrix containing discrete locations, e.g., encapsulated pockets, filled with the therapeutic agents throughout the body 121 of the implantable device 120. In some cases, the polymer matrix can include two or more biodegradable polymers such that at least a portion of the body 121 degrades at a different rate, or over a different time frame, when compared to another portion of the body 121.

Still referring to FIG. 1, the implantable device 120 includes the biodegradable body 121 containing the therapeutic composition 130, which can include the carrier 142 and one or more microspheres 140. More specifically, each microsphere 140 can contain the therapeutic agent, and optionally additional amounts of the carrier 142. In some cases, the therapeutic composition 130 can include a stem cell solution in place of, or in addition to the carrier 142. In some cases, each microsphere 140 can be filled with the stem cell solution in place of, or in addition to the carrier 142.

The composition 130 may include various suitable biocompatible carriers and microspheres 140 containing the therapeutic agent, e.g., stem cells. In some cases, the composition 130 can include a suspension of the microspheres 140 in the carrier 142 (or stem cell solution). In some cases, a suitable weight percentage of the components of the composition (e.g., microspheres 140 and carrier 142) yields a solution having a viscosity approximate to the viscosity of blood. Blood has a viscosity of about 3 to 4 centipoise (cP) at a temperature of 37° C.

In some cases, the composition 130 provided herein includes one or more microspheres 140 (including its contents) in an amount ranging from about 10% to about 90% by weight of the composition and the carrier 142 in an amount ranging from about 90% to about 10% by weight of the composition. In some cases, the composition 130 includes one or more microspheres 140 in an amount ranging from about 10% to about 50% by weight of the composition and the carrier 142 in an amount ranging from about 50% to about 90% by weight of the composition. In some cases, the composition 130 includes the microspheres 140 in an amount ranging from about 1% to about 20% by weight of the composition and the carrier 142 in an amount ranging from about 80% to about 99% by weight of the composition. Preferably, in some cases, the composition 130 includes the microspheres 140 in an amount ranging from about 30% to about 50% by weight of the composition and the carrier in an amount ranging from about 50% to about 70% by weight of the composition. More preferably, in some cases, composition 130 includes the microspheres 140 in an amount of about 40% by weight of the composition and the carrier in an amount of about 60% by weight of the composition.

The implantable device 120 provided herein can deliver the composition 130 containing microspheres 140, in which each microsphere 140 includes a wall containing a biodegradable polymer membrane, and a therapeutic agent encapsulated by the wall. The polymer membrane (or also referred to as a “shell”) encapsulates the therapeutic agent contained within each microsphere 140. Encapsulation of the therapeutic agent can allow for its controlled release and protection from degradation. In some cases, the walls of the microspheres 140 can include a nonporous polymer layer to suitably store the therapeutic agents within the body 121. The nonporous polymer layer can help to retain the therapeutic agents within the microsphere 140 in some cases, and thus prevent the therapeutic agents from being prematurely released from the microspheres 140. Each microsphere 140 optionally encapsulates a suitable carrier 142 (e.g., a liquid carrier) described herein for forming a suspension containing the therapeutic agent. In some cases, the liquid carrier 142 suspending the microspheres 140 and the liquid carrier 142 suspending the therapeutic agent within each microsphere 140 are substantially equivalent, substantially similar, or different from one another.

Each microsphere 140 can include a range of suitable weight percentages of the polymer membrane and the therapeutic agent. In some cases, the weight percentage of the therapeutic agent 140 can be configured to deliver a suitable volume of therapeutic agent per microsphere 140. In some cases, the weight percentage of the polymer membrane may be configured to provide the microsphere 140 with suitable structural stability prior to its degradation, as well as a suitable degradation time in which the polymer membrane of the microsphere 140 disintegrates and allows release of the therapeutic agent from the interior of the microsphere 140. For example, in some cases, each microsphere 140 includes the polymer membrane in an amount ranging from about 2% to about 50% by weight (e.g., from about 2% to about 30%, from about 5% to about 30%, from about 10% to about 30%, or from about 20% to about 30% by weight) of the composition. In some cases, each microsphere 140 includes the therapeutic agent in an amount ranging from about 50% to about 98% by weight (e.g., from about 50% to about 90%, from about 70% to about 90%, from about 80% to about 90%, from about 50% to about 80% by weight) of the composition.

In some cases, the microsphere 140 can include a microsphere composition containing a range of suitable weight percentages of the polymer membrane, the therapeutic agent, and a carrier 142 (e.g., saline). In some cases, the weight percentages of the therapeutic agent and the carrier 142 within each microsphere 140 are configured to achieve a solution (e.g., suspension) with a viscosity approximate to the viscosity of blood. For example, in some cases, each microsphere 140 includes a microsphere composition containing the therapeutic agent in an amount ranging from about 10% to about 90% by weight of the microsphere composition and the carrier in an amount ranging from about 90% to about 10% by weight of the microsphere composition. In some cases, the microsphere 140 includes the therapeutic agent in an amount ranging from about 10% to about 50% by weight of the microsphere composition and the carrier in an amount ranging from about 50% to about 90% by weight of the microsphere composition. In some cases, the microsphere composition includes the therapeutic agent in an amount ranging from about 1% to about 20% by weight of the microsphere composition and the carrier in an amount ranging from about 80% to about 99% by weight of the microsphere composition. Preferably, in some cases, the microsphere composition includes the therapeutic agent in an amount ranging from about 30% to about 50% by weight of the microsphere composition and the carrier in an amount ranging from about 50% to about 70% by weight of the microsphere composition. More preferably, in some cases, microsphere composition includes the therapeutic agent in an amount of about 40% by weight of the microsphere composition and the carrier in an amount of about 60% by weight of the microsphere composition.

As used herein, microspheres 140 can include various shapes including, but not limited to, a spherical shape, or a cylindrical shape. The microspheres 140 of the therapeutic composition provided herein can have a range of suitable diameters. For example, in some cases, the diameter of the microspheres 140 can range from about 10 microns to about 5,000 microns (e.g., from about 20 microns to about 2,000 microns, from about 50 microns to about 1,000 microns, from about 100 microns to about 500 microns, from about 200 microns to about 400 microns, from about 10 microns to about 50 microns, from about 20 microns to about 70 microns, from about 50 microns to about 100 microns, from about 70 microns to about 150 microns, from about 100 microns to about 200 microns, from about 150 microns to about 350 microns, from about 200 microns to about 500 microns, from about 500 microns to about 1,000 microns, from about 1,000 microns to about 2,000 microns, from about 2,000 microns to about 5,000 microns, from about 100 microns to about 300 microns, from about 200 microns to about 300 microns, from about 250 microns to about 400 microns, or from about 200 microns to about 5,000 microns) before an implantation. In some cases, the average diameter of the microspheres may be about 10 microns (e.g., about 50 microns, about 100 microns, about 200 microns, about 250 microns, about 300 microns, about 400 microns, about 500 microns, about 1 mm, about 2 mm, or about 5 mm).

The microspheres 140 provided herein can be sized to a range of suitable volumes. In some cases, each microsphere 140 can be sized to contain from about 0.1 ml to about 2 ml volume of fluid (e.g., from about 0.1 ml to about 1.5 ml, from about 0.2 ml to about 1.2 ml, from about 0.3 ml to about 1.0 ml, from about 0.5 ml to about 0.7 ml, from about 0.1 ml to about 0.3 ml, from about 0.3 ml to about 0.5 ml, from about 0.5 ml to about 0.7 ml, from about 0.7 ml to about 1.0 ml, from about 1.0 ml to about 1.5 ml, or from about 1.5 ml to about 2.0 ml).

The wall of each microsphere 140 provided herein can include the polymer membrane made of a biodegradable polymer for encapsulating the therapeutic agent. The polymer membrane of the microspheres 140 provided herein can have any suitable thickness. A suitable thickness can be based on one or more factors, for example, a particular therapeutic treatment type, a treatment dosage rate, and the patient's health. In some cases, the thickness of the polymer membrane is proportional to the microsphere diameter. For example, the polymer membrane thickness can range from about 5% to about 20% of the microsphere diameter. In some cases, the thickness of the polymer membrane can range from about 0.5 microns to about 1,000 microns (e.g., from about 0.5 microns to about 750 microns, from about 1 microns to about 500 microns, from about 5 microns to about 250 microns, from about 10 microns to about 100 microns, from about 25 microns to about 50 microns, from about 0.5 microns to about 1 micron, from about 1 micron to about 10 microns, from about 10 microns to about 50 microns, from about 50 microns to about 100 microns, from about 100 microns to about 500 microns, or from about 500 microns to about 1,000 microns).

The therapeutic composition provided herein can include microspheres 140 with a suitable rate of degradation of the polymer membrane to release the therapeutic agent contained therein. The rate of degradation may be varied to achieve a desired degradation time, where the degradation time is an amount of time in which the polymer membrane of the microsphere 140 disintegrates and allows release of the therapeutic agent from the interior of the microsphere 140. In some cases, the degradation time can range from about 5 minutes to about 72 hours (e.g., from about 15 minutes to about 60 hours, from about 30 minutes to about 48 hours, from about 60 minutes to about 24 hours, from about one hour to about 24 hours, from about 2 hours to about 12 hours, from about 3 hours to about 6 hours, from about 5 minutes to about 30 minutes, from about 30 minutes to about 60 minutes, from about 1 hour to about 3 hours, from about 3 hours to about 6 hours, from about 6 hours to about 12 hours, from about 12 hours to about 24 hours, from about 24 hours to about 48 hours, from about 1 hour to about 48 hours, from about 6 hours to about 24 hours, from about 12 hours to about 24 hours, from about 24 hours to about 48 hours, or from about 48 hours to about 72 hours).

The therapeutic composition 130 provided herein can include microspheres 140 that include a suitable material for the biodegradable polymer membrane. In some cases, a suitable biodegradable polymer membrane includes materials susceptible to enzymatic degradation in the patient's blood. Exemplary materials of the biodegradable polymer membrane can include, without limitation, polylactic acid (PLA), polyglycolic acid (PGA), copolymers of PLA and PGA, poly-L-lactide (PLLA), poly-D,L-lactide (PDLA), poly-capralactone (PCL), and combinations thereof.

The therapeutic composition 130 can contain various suitable therapeutic agents. Exemplary therapeutic agents can include, without limitation, stem cells, adenoviruses, chemotherapeutic agents, immunosuppressants, proteins, nucleic acids, and combinations thereof. Stems cells can be useful for tissue reconstruction, regeneration and/or repair. Exemplary stem cells can include, without limitation, mesenchymal stem cells that are isolated from adult tissue, induced pluripotent stem cells (iPS cells or iPSCs), embryonic stem cells, and combinations thereof. In some cases, the therapeutic agent includes an effective amount of a protein, such as a hematopoietic progenitor cell antigen CD34 that purportedly activates the immune system. In some cases, the therapeutic agent can include additional ingredients, such as a freezing media, for example, for preserving the therapeutic agent.

The therapeutic compositions 130 provided herein can be used to deliver therapeutic agents for the treatment of various diseases. The therapeutic compositions 130 provided herein may also deliver therapeutic agents to various locations within the patient's body that include, but are not limited to, the renal system, or the vascular system within brain, heart, or limbs. For example, the therapeutic compositions 130 provided herein can deliver one or more therapeutic agents to a targeted vascular region and provide a controlled release over time to treat the targeted areas.

In some cases, the therapeutic composition 130 provided herein can include a liquid carrier 142 containing a first microsphere 140a (or a first plurality of microspheres), and a second microsphere 140b (or a second plurality of microspheres), wherein the first microsphere 140a has a different composition and/or a different characteristic (e.g., release profile) than the second microsphere 140b. For example, in some cases, the first microsphere 140a can include a polymer membrane having a different degradation rate (e.g., a faster or slower degradation rate) than a polymer membrane of the second microsphere 140b. In some cases, the polymer membranes of the first microspheres 140a can include a different material than the polymer membranes of the second microspheres 140b. In some cases, the polymer membranes of the first and second microspheres 140a, 140b can be made of different types of polymers, copolymers that include different monomer units, or copolymers having different ratios of at least two monomers. For example, the biodegradable polymer wall of the first microspheres 140a may generally degrade faster or slower than the biodegradable polymer wall of the second microspheres 140b, in some cases.

In some cases, the polymer membrane of the first microsphere 140a can have a different thickness than the polymer membrane of the second microsphere 140b to produce at least two or more microspheres 140 having different degradation times. In some examples, the polymer membrane of the first microsphere 140a has a thinner wall than the polymer membrane of the second microsphere 140b such that the first microsphere 140a will have a shorter degradation time than the second microsphere 140b. In some examples, the polymer membranes of the first microspheres 140a may be generally thicker than the polymer membranes of the second microspheres 140b such that the first microsphere 140a will have a longer degradation time than the second microsphere 140b. Accordingly, the first microsphere 140a may be configured to release therapeutic agents faster or slower than the second microsphere 140b based on the thickness ratio of the polymer membrane of the first microsphere 140a relative to the second microsphere 140b. For example, in some cases, the ratio of the average wall thickness of the first microspheres 140a relative to the second microspheres 140b can be about 1:1, 1:2, 1:3, 1:4, 1:5, 5:1, 4:1, 3:1, or 2:1. In some cases, the ratio of the average wall thickness of the first microsphere 104a relative to the second microsphere 140b can range from about 1:1 to about 1:5 (e.g., from about 1:1 to about 1:4, from about 1:1 to about 1:3, from about 1:1 to about 1:2, from about 1:2 to about 1:4, from about 1:2 to about 1:3, from about 1:3 to about 1:4, or from about 1:4 to about 1:5). In some cases, the ratio of the average wall thickness of the first microsphere 104a relative to the second microsphere 140b can range from about 5:1 to about 1:1 (e.g., from about 5:1 to about 2:1, from about 5:1 to about 3:1, from about 5:1 to about 4:1, from about 4:1 to about 1:1, from about 4:1 to about 2:1, from about 4:1 to about 3:1, from about 3:1 to about 1:1, or from about 3:1 to about 2:1, or from about 2:1 to about 1:1).

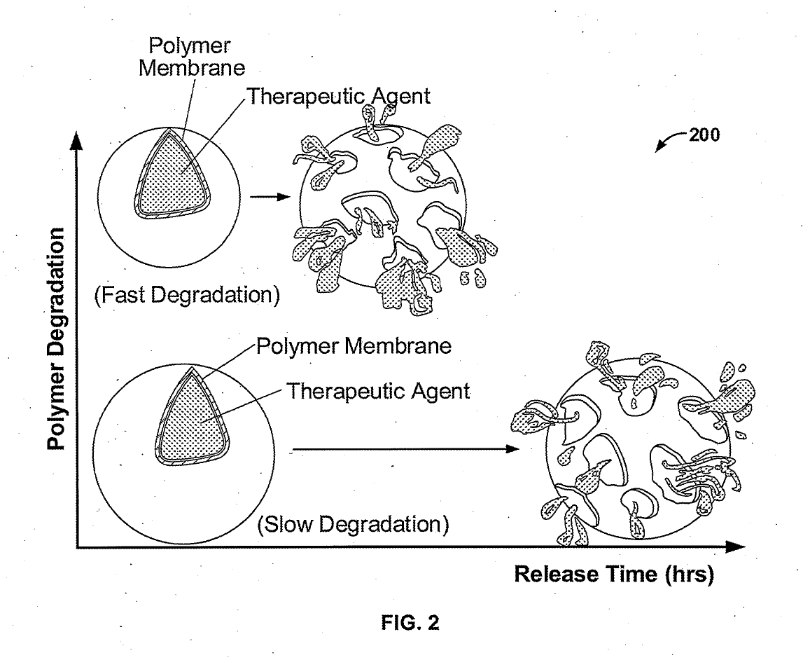

FIG. 2 is a graph 200 illustratively comparing two different exemplary microspheres (e.g., a slow-release microsphere and a fast-release microsphere) that have different degradation times. Certain embodiments of the compositions provided herein include at least two different types of microspheres, where each microsphere type has a different degradation rate. As discussed herein, compositions can include fast-release microspheres and slow-release microspheres, in which fast-release microspheres have a faster average degradation rate or shorter average time to degrade, as compared to the average degradation rate or degradation time of the slow-release microspheres. As depicted in the figures, an exemplary fast-release microsphere can be associated to a low release time and a high degradation rate of the polymer (outer) membrane while an exemplary slow-release microsphere is associated with an increased release time and a decreased degradation rate of the polymer membrane. The benefit of the therapeutic composition with variable-release microspheres includes allowing a medical practitioner to deliver to the patient during a single intravascular procedure a treatment that provides multiple therapeutic agent exposures over a given period of time.

The therapeutic compositions provided herein can be made with microspheres having a suitable fast-release degradation time (or rate) and a suitable slow-release degradation time (or rate). For example, in some cases, the fast-release microspheres can degrade within 24 hours (e.g., within 1 hour, 3 hours, 6 hours, 9 hours, 12 hours, or 24 hours) and the slow-release microspheres can degrade between 24 hours and 72 hours (e.g., 25 hours, 30 hours, 40 hours, 48 hours, 54 hours, 60 hours, or 72 hours). In some cases, the fast-release microspheres can degrade within 1 week (e.g., within 1 day, 2 days, 3 days, 4 days, 5 day, 6 days, or 7 days) and the slow-release microspheres can degrade from about 1 week to about 1 month (1 week, 2 weeks, 3 weeks, 4 weeks, 1 month). In some cases, slow-release microspheres degrade about 1 hour to about 1 month (e.g., about 1 hour, about 2 hours, about 3 hours, about 6 hours, about 12 hours, about 24 hours, about 48 hours, about 54 hours, about 60 hours, about 72 hours) after the fast-release microspheres have degraded. In some cases, the slow-release microspheres can have a degradation rate that is about 1.1, about 1.2, about 1.5, about 2, about 3, about 4, about 5, about 10, about 20, or greater than 20 times greater than the degradation rate of the fast-release microspheres.

The therapeutic compositions provided herein can be made with one or more carriers having a suitable fast-release degradation time (or rate) and a suitable slow-release degradation time (or rate). For example, in some cases, the fast-release carrier can degrade within 24 hours (e.g., within 1 hour, 3 hours, 6 hours, 9 hours, 12 hours, or 24 hours) and the slow-release carrier can degrade between 24 hours and 72 hours (e.g., 25 hours, 30 hours, 40 hours, 48 hours, 54 hours, 60 hours, or 72 hours). In some cases, the fast-release carrier can degrade within 1 week (e.g., within 1 day, 2 days, 3 days, 4 days, 5 day, 6 days, or 7 days) and the slow-release carrier can degrade from about 1 week to about 1 month (1 week, 2 weeks, 3 weeks, 4 weeks, 1 month). In some cases, slow-release carrier degrade about 1 hour to about 1 month (e.g., about 1 hour, about 2 hours, about 3 hours, about 6 hours, about 12 hours, about 24 hours, about 48 hours, about 54 hours, about 60 hours, about 72 hours) after the fast-release carrier have degraded. In some cases, the slow-release carrier can have a degradation rate that is about 1.1, about 1.2, about 1.5, about 2, about 3, about 4, about 5, about 10, about 20, or greater than 20 times greater than the degradation rate of the fast-release carrier.

Certain embodiments of the therapeutic composition provided herein contain at least two or more microspheres containing different intra-microsphere materials. For example, in some cases, the therapeutic composition provided herein can include first microspheres containing a different therapeutic agent than the therapeutic agent of the second microspheres. In some cases, the first and second microspheres can contain different concentrations of the same therapeutic agent.

In some embodiments, the device provided herein contains a composition that includes multi-encapsulated microspheres, in which at least one (smaller) microsphere is encapsulated within another (larger) microsphere. Certain embodiments of the therapeutic composition provided herein include multi-encapsulated microspheres that allow for a controlled, prolonged delivery of a therapeutic agent. The outer microsphere can include a wall made of the biodegradable polymer membrane, in which the wall envelopes a therapeutic agent optionally suspended in a carrier, and at least one smaller (inner) microsphere. The polymer membrane of the outer microsphere encapsulates the therapeutic agent and the inner microsphere contained within the outer microsphere. Encapsulation of the therapeutic agent within the outer microsphere can allow for the controlled release of the therapeutic agent and inner microsphere contained therein. The inner microsphere also includes a wall made of the biodegradable polymer membrane, and a therapeutic agent optionally suspended in a carrier. The inner microsphere encapsulates the therapeutic agent and allows for a prolonged, staged release of a therapeutic agent. In some cases, the outer microsphere can include more than one inner microsphere (e.g., two or more microspheres, three or more microspheres, four or more microspheres, five or more microspheres, ten or more microspheres, twenty or more microspheres, thirty or more microspheres, forty or more microspheres, or fifty or more microspheres). For example, in some cases, the therapeutic composition provided herein can include two inner microspheres; a first inner microsphere that encapsulates a second inner microsphere. In some cases, the therapeutic composition provided herein can include three, four, five, or more than five inner microspheres, where each microsphere is encapsulated within another microsphere with exception of the smallest microsphere.

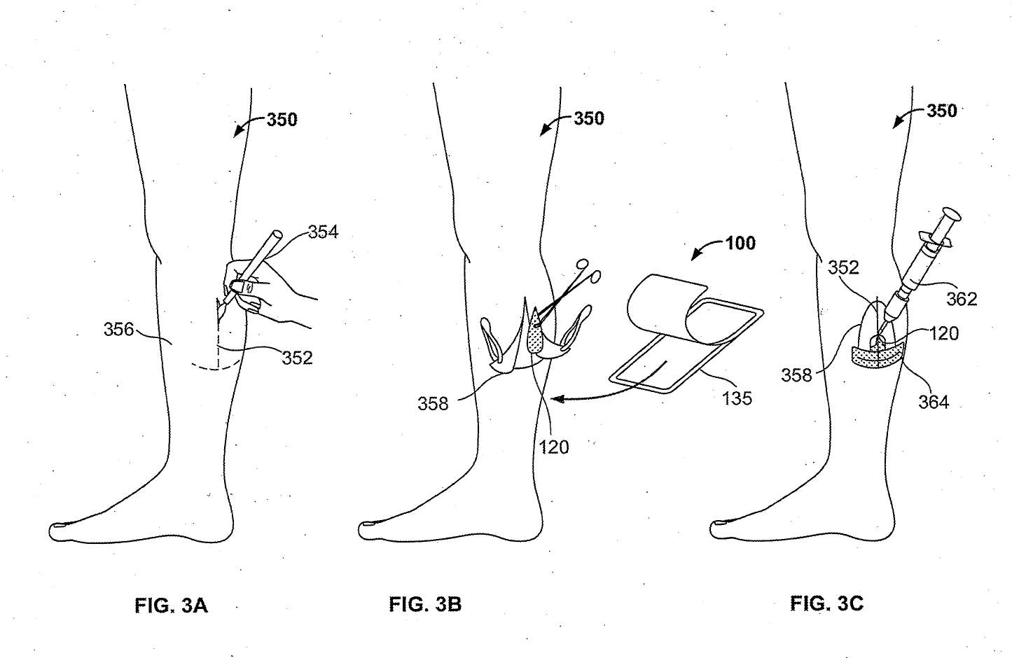

Referring to FIGS. 3A-3C, the system 100 of FIG. 1 can be used to introduce the implantable device into the patient's body 350 using a surgical dissection technique and using local anesthetics. As shown in FIG. 3A, an incision 352 can be made in the skin by using a dissection instrument 354 (e.g., a scalpel) at a desired location, for example, the calf portion of a leg. In some cases, one or more dissection instruments can be used to dissect targeted tissue. In some cases, a cauterization device (not shown) may be used in conjunction with the dissection instrument to cauterize vessels as desired to minimize fluid loss during a medical procedure. The dissection instrument can be used to cut through the epidermis, the dermis, and/or subcutaneous fat portion of the tissue.

Referring to FIG. 3B, multiple incisions can be made to the skin and tissue such that portions of the incised tissue 358 may be pulled back to enable positioning of the implantable device 120 within the patient 350. Once the tissue 358 has been incised, the implantable device can be removed from its sterile packaging 135 and placed in the desired implantation site.

Referring to FIG. 3C, once the device 120 has been positioned at the desired implantation location, the tissue 358 surrounding the incision(s) 352 can be restored back to its original position, thus covering the device 120. In some cases, as shown, a sutureless closure (e.g., closure by applying liquid surgical adhesive 362 or adhesive strips 364) can be used to secure adjacent tissue portions together. Over a predetermined time frame, the implanted device 120 will degrade and release the microspheres, or pockets filled with the therapeutic agent, into the patient's body 350.

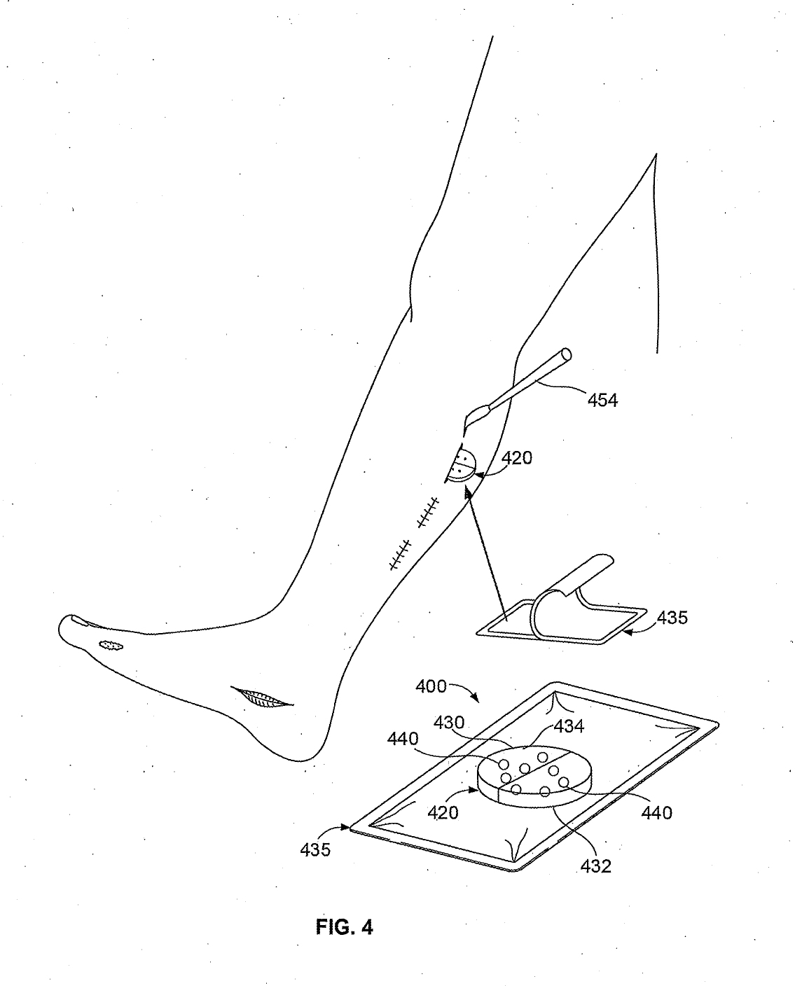

Referring to FIG. 4, an alternative embodiment of the system 400 provided herein can include a disc-shaped implantable device 420 enclosed within sterile or sterilizable packaging 435. The implantable device 420 can include a body 430 and discrete locations (e.g., pockets) within the body 430 that contain a predetermined volume of a therapeutic agent. The body 430 of the device 420 can include a carrier, such as a gel or polymer matrix, that encapsulate the pockets of therapeutic agent 440 therein.

In some cases, the implantable device 420 can include a disc-shaped body have outer dimensions that including a diameter of about 20 mm and a width of about 0.5 mm. In some cases, the body 430 can have a wall thickness of about 3 mm. The implantable device 420 can be made into any suitable shape, e.g., an oval-shape, a polygonal shape (e.g., a square, rectangular, triangular shape), or an irregular or asymmetrical shape.

Certain embodiments of the implantable device 420 can include two or more carriers 430 comprising a liquid, gel, or a polymer matrix to facilitate multiple release of a therapeutic agent 440 within the patient's body. In some cases, the implantable device 420 includes a first region 432 containing a first carrier encapsulating one or more discrete locations containing a therapeutic agent within the first region 432, and a second region 434 containing a second carrier encapsulating one or more discrete locations containing the therapeutic agent within the second region 434. In some cases, the first carrier contains a first biodegradable material having a different degradation rate than the second carrier material that contains a second biodegradable material. In some cases, the body 430 includes a total carrier composition that includes the first carrier in an amount ranging from about 40% to about 60% by weight of the total carrier composition and the second carrier in an amount ranging from about 40% to about 60% by weight of the total carrier composition. In some cases, the implantable device 420 includes a composition that includes the therapeutic agent provided herein in an amount ranging from about 10% to about 90% by weight of the composition and the first and second carriers in an amount ranging from about 90% to about 10% by weight of the composition.

In some cases, the implantable device provided herein can be inserted into openings created by one or more tissue incisions. The incision may be sized to allow the implantable device to pass through the skin and into the underlying tissue at a desired depth within the patient's body. In some cases, the dissection instrument 454 (or by another instrument, or by surgical finger manipulation) may be used to create a tissue pocket under the skin for receiving the implantable device in the underlying tissue. Following device insertion, the incision may be closed by suturing adjacent tissue portions together.

### Methods of Manufacturing

There are a number of processes available to manufacture the implantable devices provided herein. Exemplary processes, which can depend on particular materials used, can include, but are not limited to, phase separation or precipitation processes, emulsion/solvent evaporation processes, spraying processes, extrusion processes, injection molding processes, injection or microinjection processes, sphere blowing processes, electrospinning processes, 3D printing, and combinations thereof. Processes involving injections for filling the microspheres provided herein, can optionally include a heating step for sealing one or more injection site(s).

In some cases, the microspheres of the therapeutic composition provided herein can be made by using a microparticle preparation technique, as described in the following reference: Makadia, H. K., and Siegel, S. J. Poly Lactic-co-Glycolic Acid (PLGA) as Biodegradable Controlled Drug Delivery Carrier, Polymers (Basel); 3(3): 1377-1397; 2011.

Some exemplary manufacturing processes for creating microspheres of the therapeutic composition provided herein include using an immiscible solution process, or emulsion and solvent evaporation process. For example, microspheres can be made using a solvent evaporation and solvent extraction process as described by Jain R. A., The manufacturing techniques of various drug loaded biodegradable poly(lactide-co-glycolide) (PLGA) devices. Biomaterials; 21:2475-2490; 2000 (“the Jain reference”).

In some cases, the immiscible solution process can include formulating a solution to form the polymer membrane (i.e., shell). In some cases, the immiscible solution process can include formulating a solution to form the polymer membrane (i.e., shell). Exemplary solutions may be formed by dissolving a desired amount of a polymer solution (e.g., PLGA and/or PLLA in a solvent, such as dimethylformamide (DMF)). An immiscible therapeutic agent may be added to the polymer membrane solution to form spheres within the solution. Any excess solution can subsequently be removed to yield microspheres that include the therapeutic agent coated in a solidified polymer membrane made from the shell solution.

Certain exemplary manufacturing processes for creating microspheres of the therapeutic composition provided herein can use a “bubble-forming” method. For example, a thin layer of a soft biodegradable material (e.g., a polymer such as PLGA or PLLA) may be injected with a therapeutic agent using, for example, a needle injector, to form a pocket (or bubble) filled with the therapeutic agent inside the layer of the biodegradable material. In some cases, the therapeutic agent can be added until the layer of the biodegradable stretches to form walls of the microspheres described herein. The injection site(s) (i.e., puncture sites) of the microsphere can be sealed by applying heat at a temperature near or at the glass transition temperature, or the melt temperature, of the layer of the biodegradable material to form a microsphere that encapsulates the therapeutic agent within the layer of the biodegradable material.

Some exemplary manufacturing processes for creating microspheres of the therapeutic composition provided herein may include using an extrusion method. In some cases, a material (e.g., PLGA) may be extruded through an extrusion dye to form a microtube or a micropellet. Each microtube or micropellet can be filled with a therapeutic agent by, for example, injecting the therapeutic agent into a center portion of the microtube or micropellet with a needle injector. In some cases, injected microtubes may be cut and sealed simultaneously to form cylinder-shaped vesicles that are filled with the therapeutic agent.

In some cases, the microspheres provided herein can be made by using a spraying process. For example, a therapeutic agent may be dispersed as droplets on a polymer film (e.g., a PLGA film). After droplets of the therapeutic agent have been placed onto the polymer film, a polymer membrane solution can be sprayed over the droplets to form microspheres that include the polymer membrane which encapsulates the droplets of the therapeutic agent. In some cases, the spray drying process as well as other types of microsphere forming processes (e.g., double emulsion process or a phase separation process) as described by the Jain reference, may be used to create microspheres provided herein. See id. at 2478-2480.

Some embodiments of the microspheres provided can be made using a molding process and an injection process. For example, in some cases, a mold can be injected with a suitable polymer material provided herein to create the wall of the microsphere. Once the microsphere wall has been completed, the interior hollow region may be filled with a therapeutic agent using an injector that pierces the wall in one or multiple locations. The pierced locations can optionally be sealed by using a heating process that allows the polymer membrane wall to reflow and fill any punctures.

In some cases, 3D printing can be used to form the microspheres provided herein. For example, a first nozzle of a 3D printer may be used to form the wall of a first microsphere while a second nozzle of the 3D printer fills the interior cavity of the first microsphere with a therapeutic agent. Optionally, the 3D printer can be used to form the wall and fill the interior cavity of a second microsphere within the first microsphere. In some cases, the 3D printer may be used to form wall of a hollow (empty) microsphere such that the interior hollow region can be filled with a therapeutic agent by an injector that pierces the wall in one or more locations. An optional heating process may be applied following the injection process to seal any puncture sites.

Some embodiments of the microspheres can be made using an electrospinning process. For example, one or more droplets of a therapeutic agent can be placed on a base plate (or flat surface) at a desired contact angle (e.g., at a 147 angle relative to a horizontal plane) to promote movement of the droplet. An electrospun material can be disposed onto the droplet as it rolls along the flat surface to form a microsphere in which the electrospun material forms the polymer wall that encapsulates the therapeutic droplet.

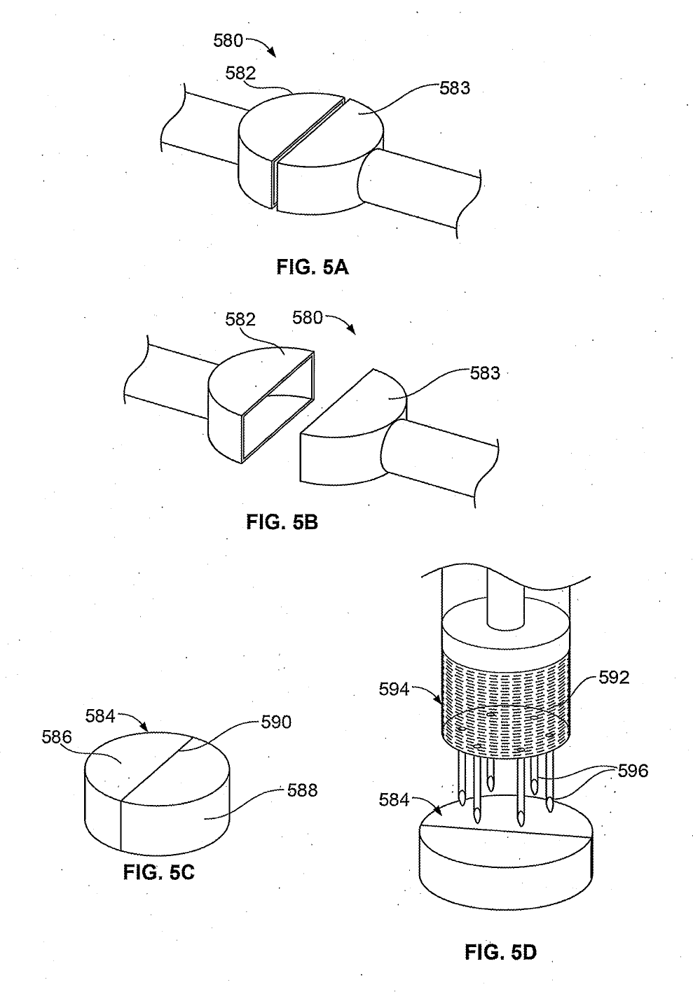

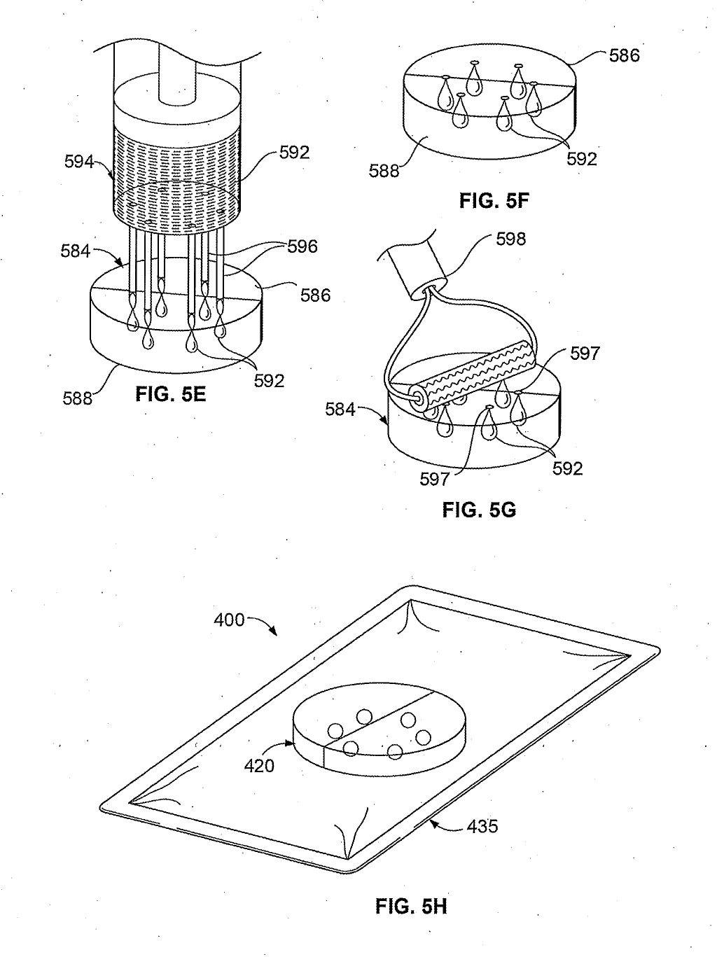

FIGS. 5A-5H are schematic illustrations showing an exemplary method of manufacturing of the implantable device provided herein. The exemplary method can use a combination of various processes that may include injection molding, injection loading of the therapeutic agent, heat sealing, and application of a sterilizable package.

An exemplary method of manufacturing an implantable device can include forming a first portion of a preformed body containing a first biodegradable material and forming a second portion of the preformed body containing a second biodegradable material. The second biodegradable material of the preformed body can have a different degradation rate than the first biodegradable material. In some cases, the forming of the first portion or second portion, or both, can include injection molding, phase separation, emulsion or solvent evaporation, spraying, extrusion, sphere blowing, or 3D printing. In method can include joining the first and second portions of the preformed body together to form the device. The method can include injecting a therapeutic agent into the first and second portions of the device. The method can include optionally encapsulating the therapeutic agent within the first and second portions of the device by sealing the injection sites created during the injecting step.

Referring to FIGS. 5A-5C, a preformed component 584 (e.g., a disc-shaped component; see FIG. 5C) of the implantable device may be formed by injection molding. The performed component 584 can be made into any suitable shape, e.g., a polygonal shape such as a square, rectangular, triangular shape, as well as an irregular or asymmetrical shape. An exemplary injection molding equipment 580 can include two mold components 582, 583 that can come together to form an interior cavity, as shown in FIGS. 5A and 5B. When the mold components 582, 583 are in a closed state (FIG. 5A), an injectant (e.g., a molten plastic material or an uncured thermoset) can be introduced into the cavity and subsequently cooled to form a molded preform component 584 shown in FIG. 5C. In some cases, preformed component 584 of the implantable device can be formed by other types of processing methods, such as material formation by phase separation or precipitation, emulsion/solvent evaporation, spraying methods, extrusion methods, sphere blowing, 3D printing (i.e. additive manufacturing), or combinations thereof.

Referring to FIG. 5C, certain embodiments provided herein include a preformed component 584 made of at least two different materials having different degradation rates. In some cases, a first portion 586 of the preformed component includes a first material and a second portion 588 of the preformed component 584 includes a second material, wherein the first and second materials comprise different compositions or materials that allow the first portion 586 to degrade at a different rate than the second portion 588. The first and second materials can be injected into mold at opposite ends of the molding cavity of the injection molding equipment such that the two materials are joined together at an interface 590 but the two materials do not blend together.

Referring to FIGS. 5D-5F, the therapeutic agent(s) 592 can be added to the preformed component 584 to form the implantable device provided herein using an injection or a microinjection method. As shown in FIGS. 5D and 5E, an injector 594 with one or more needle tips 596 can be used to penetrate the preformed component 584 with a predetermined penetration depth. As illustrated in FIG. 5E, the therapeutic agent 592 can be injected into multiple locations within the preformed component 584 to disperse drops of the therapeutic agent 592 into discrete pockets throughout the body of the preformed component 584.

Referring to FIG. 5G, the preformed component 584 can be optionally subjected to a heat application process to form the implantable device provided herein. As shown, a hot roller 598 can be applied to one or more surfaces of the preformed component 584 to seal any puncture sites 597 left behind by the injection process method. In some cases, preformed component 584 of the implantable device can be heat sealed using other types of processing methods, such as material formation by phase separation or precipitation, emulsion/solvent evaporation, spraying methods, extrusion methods, sphere blowing, 3D printing, or combinations thereof.

Referring to FIG. 5H, the implantable device 420 can be enveloped in the sterile or sterilizable packaging 435. In some cases, the sterile packaging 435 can include a sealable bag or envelope capable of withstanding sterilization conditions.

It should be understood that one or more design features of the embodiments provided herein can be combined with other features of other embodiments provided herein. In effect, hybrid designs that combine various features from two or more of the device designs provided herein can be created, and are within the scope of this disclosure.

While this specification contains many specific implementation details, these should not be construed as limitations on the scope of the disclosure or of what may be claimed, but rather as descriptions of features that may be specific to particular embodiments of particular disclosures. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

In addition to being directed to the teachings described above and claimed below, systems, devices, and methods having different combinations of the features described above and claimed below are contemplated. As such, the description is also directed to other devices and/or methods having any other possible combination of the dependent features claimed below.

Numerous characteristics and advantages have been set forth in the preceding description, including various alternatives together with details of the structure and function of the devices and/or methods. The disclosure is intended as illustrative only and as such is not intended to be exhaustive. It will be evident to those skilled in the art that various modifications may be made, especially in matters of structure, materials, elements, components, shape, size and arrangement of parts including combinations within the principles of the present disclosure, to the full extent indicated by the broad, general meaning of the terms in which the appended claims are expressed. To the extent that these various modifications do not depart from the spirit and scope of the appended claims, they are intended to be encompassed therein. All references, publications, and patents referred to herein, including the figures and drawings included therewith, are incorporated by reference in their entirety.

What is claimed is:

1. An implantable medical device comprising a body that includes a flexible outer layer encapsulating a composition, the composition comprising:
(i) one or more first microspheres, each first microsphere containing a first therapeutic agent and a wall comprising a biodegradable polymer that encapsulates the first therapeutic agent;
(ii) one or more second microspheres, each second microsphere containing a second therapeutic agent and a wall comprising a biodegradable polymer that encapsulates the second therapeutic agent; and
(iii) a carrier.

2. The implantable medical device of claim 1, wherein the body includes an oval-shaped or disc-shaped pouch.

3. The implantable medical device of claim 1, wherein the outer layer of the body comprises a biodegradable polymer including polylactic acid (PLA), polyglycolic acid (PGA), a copolymer of PLA and PGA, poly-L-lactide (PLLA), poly-D,L-lactide (PDLA), poly-capralactone (PCL), or a combination thereof.

4. The implantable medical device of claim 1, wherein the carrier comprises a liquid carrier.

5. The implantable medical device of claim 4, wherein the liquid carrier comprises water, saline solution, a serum, or a combination thereof.

6. The implantable medical device of claim 1, wherein the composition comprises the first and second microspheres in an amount ranging from about 10% to about 90% by weight of the composition and the carrier in an amount ranging from about 90% to about 10% by weight of the composition.

7. The implantable medical device of claim 1, wherein the composition comprises the first microspheres, the second microspheres, or a combination of both, in an amount ranging from about 10% to about 50% by weight of the composition, and the carrier in an amount ranging from about 50% to about 90% by weight of the composition.

8. The implantable medical device of claim 1, wherein the biodegradable polymer of the first microsphere generally degrades faster or slower than the biodegradable polymer of the second microsphere.

9. The implantable medical device of claim 1, wherein the biodegradable polymer of the first microsphere, the second microsphere, or both, comprises polylactic acid (PLA), polyglycolic acid (PGA), a copolymer of PLA and PGA, poly-L-lactide (PLLA), poly-D,L-lactide (PDLA), poly-capralactone (PCL), or a combination thereof.

10. The implantable medical device of claim 1, wherein each of the first and second microspheres have a diameter ranging from about 0.2 millimeters to about 5 millimeters.

11. The implantable medical device of claim 1, wherein the walls of the first and second microspheres comprise a nonporous polymer layer.

12. An implantable medical device comprising a body including:
(i) a first region comprising a first carrier encapsulating one or more discrete locations containing a therapeutic agent within the first region;
(ii) a second region comprising a second carrier encapsulating one or more discrete locations containing the therapeutic agent within the second region;
wherein the first carrier comprises a first biodegradable material having a different degradation rate than the second carrier material comprising a second biodegradable material.

13. The implantable medical device of claim 12, wherein the first and second carriers comprise a gel, or a solid polymer matrix.

14. The implantable medical device of claim 13, wherein the gel or the solid polymer matrix comprises a biodegradable polymer including polylactic acid (PLA), polyglycolic acid (PGA), a copolymer of PLA and PGA, poly-L-lactide (PLLA), poly-D,L-lactide (PDLA), poly-capralactone (PCL), or a combination thereof.

15. The implantable medical device of claim 12, wherein the first carrier has a degradation rate that is about 1.1, about 1.2, about 1.5, about 2, about 3, about 4, about 5, about 10, or about 20 times greater than a degradation rate of the second carrier.

16. The implantable medical device of claim 12, wherein the body includes a total carrier composition that comprises the first carrier in an amount ranging from about 40% to about 60% by weight of the total carrier composition and the second carrier in an amount ranging from about 40% to about 60% by weight of the total carrier composition.

17. The implantable medical device of claim 12, wherein the composition comprises the therapeutic agent in an amount ranging from about 10% to about 90% by weight of the composition and the first and second carriers in an amount ranging from about 90% to about 10% by weight of the composition.

18. The implantable medical device of claim 12, wherein the therapeutic agent is selected from the group consisting of stem cells, adenoviruses, chemotherapeutic agents, immunosuppressants, proteins, nucleic acids, or a combination thereof.

19. A method of manufacturing an implantable device, the method comprising:
forming a first portion of a preformed body comprising a first biodegradable material;
forming a second portion of the preformed body comprising a second biodegradable material, the first portion degrading faster or slower than the second portion;
joining the first and second portions of the preformed body together to form the device;
injecting a therapeutic agent into the first and second portions of the device; and
optionally encapsulating the therapeutic agent within the first and second portions of the device by sealing the injection sites created during the injecting step.

20. The method of claim 19, wherein the forming of the first portion or the second portion comprises using one of injection molding, phase separation, emulsion or solvent evaporation, spraying, extrusion, sphere blowing, or 3D printing.
